(19) United States
(12) Patent Application Publication
(10) Pub. No.: US 2009/0247984 A1
(43) Pub. Date: Oct. 1, 2009

(54) USE OF MICRONEEDLES FOR SMALL MOLECULE METABOLITE REPORTER DELIVERY

(75) Inventors: Marcelo Lamego, Coto De Caza, CA (US); Sean Merritt, Lake Forest, CA (US); Grace Young, Newport Coast, CA (US)

Correspondence Address: KNOBBE MARTENS OLSON & BEAR LLP, 2040 MAIN STREET, FOURTEENTH FLOOR, IRVINE, CA, 92614, US

(73) Assignee: Masimo Laboratories, Inc., Irvine, CA (US)

(21) Appl. No.: 12/258,280

(22) Filed: Oct. 24, 2008

Related U.S. Application Data
(60) Provisional application No. 60/982,394, filed on Oct. 24, 2007.

(51) Int. Cl. A61M 5/32; A61M 5/00; G01N 33/66

(52) U.S. Cl. 604/506; 604/272; 436/95

(57) ABSTRACT

Systems that can include a microneedle array and a small molecule metabolic reporter are disclosed. The systems can be used to penetrate the epidermis and monitor the change in concentration of one or more metabolite or analyte.

### RELATED APPLICATION INFORMATION

This application claims priority to U.S. Provisional Application Ser. No. 60/982,394, filed on Oct. 24, 2007, which is incorporated herein by reference in its entirety for all purposes.

### BACKGROUND

1. Field

Disclosed herein are systems and methods related to transdermal delivery of compositions which can be used to determine the concentration of one or more metabolites or analytes in a biological sample. In particular, embodiments disclosed herein relate to the use of microneedles to deliver compositions transdermally that can be used to non-invasively determine blood glucose concentration levels.

2. Description of the Related Art

Identifying and understanding the risk factors associated with diabetes is invaluable for the development and evaluation of effective intervention strategies.

Lacking normal regulatory mechanisms, diabetics are encouraged to strive for optimal control through a modulated life style approach that focuses on dietary control, exercise, and glucose self-testing with the timely administration of insulin or oral hypoglycemic medications. Invasive forms of self-testing are painful and fraught with a multitude of psychosocial hurdles, and are resisted by most diabetics. Alternatives to the currently available invasive blood glucose testing are highly desirable.

Conventional approaches to non-invasive alternatives seek to reduce or eliminate the skin trauma, pain, and blood waste associated with traditional invasive glucose monitoring technologies. In general, noninvasive optical blood glucose monitoring requires no bodily fluid samples to be withdrawn from tissue and involves external irradiation with electromagnetic radiation and measurement of the resulting optical flux (e.g. fluorescence or diffuse reflectance). One such noninvasive in vivo optical blood glucose monitoring system is disclosed in U.S. patent application Ser. No. 11/349,731, filed Feb. 7, 2006, and U.S. patent application Ser. No. 10/584,821, filed Mar. 13, 2008, which is the United States national phase entry for PCT Appl. No. PCT/US2004/043087, filed Dec. 23, 2004, which are hereby incorporated by reference in their entireties.

### SUMMARY

In some embodiments disclosed herein, there is provided a system for delivering at least one small molecule metabolic reporter (SMMR) into the human epidermis, which includes at least one SMMR and a microneedle array, wherein the microneedle array penetrates the epidermis to facilitate delivery of the SMMR.

In an embodiment, there is provided an array of solid silicon microneedles,

wherein the tips of each microneedle are comprised of at least one SMMR. In another embodiment, there is provided an array of hollow silicon microneedles, wherein each microneedle is filled with SMMR which is subsequently injected into the epidermis.

Some embodiments are directed to an in vivo method for delivering at least one SMMR into the human epidermis that can include contacting the SMMR with a microneedle array and penetrating the epidermis with the array to a depth of about 10 μm, wherein the depth corresponds with the bottom of the dead stratum corneum layer, to about 175 μm, wherein the depth corresponds with the top of the dermal layer.

Other embodiments are directed to an in vivo method for monitoring the concentration of one or more metabolite or analyte that can include contacting at least one SMMR with a microneedle array, penetrating the epidermis with the microneedle array to a depth of about 10 μm, wherein the depth corresponds with the bottom of the dead stratum corneum layer, to about 175 μm, wherein the depth corresponds with the top of the dermal layer, and monitoring a change in the concentration of the one or more metabolite or analyte in a metabolic pathway by detecting changes in the at least one SMMR at one or more time points using an optical reader. In some embodiments, the one or more metabolite or analyte may include glucose.

The use of such microneedles results in faster penetration of the SMMR and greater accuracy in glucose concentration measurement, while at the same time not requiring any invasive procedures.

These and other embodiments are described in greater detail below.

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety.

“Microneedle,” as the term is used herein, describes a needle which can penetrate the stratum corneum layer of the skin as well as the epidermis, but does not reach as far as the dermis. Through bypassing the stratum corneum, use of microneedles can effectively increase skin permeability up to 100,000-fold in some embodiments.

The terms “dermis” and “dermal layer” refer to the layer of skin between the epidermis and the subcutis.

A “small molecule metabolic reporter” is a molecule that varies fluorescence emission, absorption, and/or reflectance in relation to the concentration of one or more metabolites and/or analytes.

As used herein, a “metabolite” is a substance produced by a metabolic process, such as glycolysis, which can be quantitatively measured as an indication of the rate or quantity of a specific metabolic process.

As used herein, an “analyte” is a measurable parameter, using analytical chemistry, which can be quantitatively measured as an indication of the rate and quantity of a specific metabolic process. The term analyte is a generic term describing such concepts including but not limited to metabolites, ions, processes, conditions, physico-chemical parameters, and/or metabolic results that can be used to infer the rate or quantity of specific metabolic processes.

The phrase “molecular size attachment” refers to SMMR adducts that include, but are not limited to, structural modifications of SMMRs as the additions to the fluorescence structure of: acetoxy methyl esters, chloro-methyl derivatives, alkyl chain adducts, highly charged moieties, enzyme substrate mimics, enzyme cofactor tethers, and membrane binding tethers.

Embodiments disclosed herein are directed to a system for delivering at least one small molecule metabolic reporter (SMMR) into the human epidermis which includes at least one SMMR and an array of microneedles, wherein the array of microneedles penetrates the epidermis to facilitate delivery of the SMMR.

In some embodiments, the microneedle may have a length from approximately 50 μm to approximately 150 μm. In an exemplary embodiment, the microneedle may be approximately 50 nm in length. In some embodiments, the microneedle may penetrate skin at a depth of approximately 10 μm to approximately 175 μm. In an embodiment, the microneedle may penetrate skin at a depth approximately corresponding to the bottom of the stratum corneum. In another embodiment, the microneedle may penetrate skin at a depth approximately corresponding to the top of the dermal layer. In still other embodiments, the microneedle may penetrate skin at a depth approximately in between the bottom of the stratum corneum and the top of the dermal layer.

In some embodiments, the microneedle can be solid. In other embodiments, the microneedle can be hollow. The microneedle may be any of a variety of diameters as needed to maintain efficacy, as may be recognized by those skilled in the art. In some embodiments, the outer diameter of a microneedle can be from approximately 20 μm to approximately 100 μm. In other embodiments, the outer diameter of a microneedle can be from approximately 10 μm to approximately 50 μm. The inner diameter of a hollow microneedle can be from approximately 5 μm to approximately 70 μm in some embodiments. Those skilled in the art will appreciate that any combination of the above microneedle dimensions may be used with the systems and methods described herein.

A microneedle may be used alone or in combination with one or more additional microneedles. In some embodiments, a plurality of microneedles is arranged in an array. An array of microneedles can vary on the basis of several factors, including but not limited to, length, diameter, interneedle distance, sharpness, and the total number of microneedles used. In an exemplary embodiment, an array of microneedles may comprise a 10×10 matrix. In another embodiment, an array of microneedles may comprise a 20×20 matrix. In some embodiments, the distance between each microneedle in an array may be from approximately 100 μm to approximately 400 μm. In some embodiments, the distance between each microneedle in an array may be selected to promote even application of an SMMR while also avoiding difficulty in penetration due to the skin's elasticity. In an embodiment, the particular dimensions of the array can be chosen depending on the desired enhancement of skin permeability.

A microneedle can be manufactured from a variety of materials, including but not limited to, silicon, a metal, a polymer, and glass. In some embodiments, a microneedle may be made of silicon. Silicon microneedles, whether solid or hollow, can be etched from a silicon wafer. In some embodiments, the thickness of a silicon wafer can vary between approximately 325-375 μm for hollow microneedles and between approximately 450-550 μm for solid microneedles. In some embodiments, the volume resistivity may be approximately 10-15 Ω-cm. In an embodiment, the location of each microneedle is marked and the surrounding silicon is etched away, resulting in an array of microneedles attached to a common base.

In other embodiments, a microneedle may be made of a metal. Exemplary metals include, but are not limited to, nickel, titanium, and alloys such as stainless steel. In some embodiments, metal microneedles may be made from epoxy molds. The molds can be electroplated with a chosen metal and the epoxy can subsequently be etched away. The resulting microneedles may either be reusable or disposable. Microneedles may also be obtained from a commercial source.

SMMR properties may vary in relation to one or more metabolites and analytes. In some embodiments, the fluorescence emission, absorption, and/or reflectance of the SMMR may vary in relation to the concentration of one or more metabolites and analytes. Metabolites and analytes that may be monitored include, but are not limited to glucose, lactate, H⁺, Ca²⁺, Mg²⁺, Na⁺, K⁺, ATP, ADP, Pi, glycogen, pyruvate, NAD(P)⁺, NAD(P)H (nicotinamide adenine dinucleotide (phosphate), reduced form), FAD, FADH₂, and O₂. In an exemplary embodiment, the analyte is glucose. As a result, a variety of parameters may be measured, including but not limited to: pH (as lactate/H⁺), membrane reduction-oxidation electric potential, NAD(P)H for energy transfer, FAD⁺ (nicotinamide adenine dinucleotide, oxidized form) for energy transfer, ATP/ADP ratio, Ca²⁺-pumping rate, Mg²⁺-pumping rate, Na⁺-pumping rate, K⁺-pumping rate, and vital mitochondrial membrane stains/dyes/molecules fluorescence response. In some embodiments, in vivo glucose concentration may be determined indirectly using one of the aforementioned parameters. In other embodiments, glucose is measured directly.

The in vivo information obtained when the SMMR is brought in contact with the one or more metabolites or analytes can include, but is not limited to, assessment of metabolic function; diagnosis of metabolic disease state; monitoring and control of disease state; stress status of cells, tissues and organs; determination of vitality and viability of cells based on metabolic function; critical care monitoring; diagnosis and monitoring of cardiovascular diseases, autoimmune disorders, neurological disorders, degenerative diseases; determination of metabolic concentration; and cancer diagnosis, detection, staging and prognosis.

For example, the in vivo information obtained may provide detailed information on glucose metabolism, fructose metabolism and galactose metabolism; advanced-glycolsolated end products; and monitoring and control of diseases such as diabetes, cancer, stress and organ transplantation.

In an embodiment, one or more SMMR is selected in order to effectively determine the glucose concentration in blood for a living organism, either directly or indirectly.

A variety of SMMRs may be used with the systems and methods disclosed herein. For example, the at least one small molecule metabolic reporter can be a fluorophore, a protein labeled fluorophore, a protein comprising a photooxidizable cofactor, a protein comprising another intercalated fluorophore; a mitochondrial vital stain or dye, a dye exhibiting at least one of a redox potential, a membrane localizing dye, a dye with energy transfer properties, a pH indicating dye; a coumarin dye, a derivative of a coumarin dye, an anthraquinone dye, a cyanine dye, an azo dye, a xanthene dye, an arylmethine dye, a pyrene derivative dye, or a ruthenium bipyridyl complex dye. In some embodiments, the SMMR may be a xanthene-based boronic acid compound. In other embodiments, the SMMR may be a coumarin compound, a carbostyril compound, or derivatives thereof.

In an exemplary embodiment, the SMMR may include a compound of Formula (I):

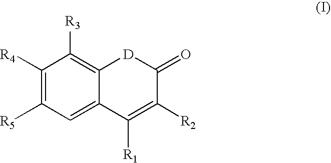

In Formula (I), D can be a heteroatom (e.g., O or N); R₁ can be selected from H, OH, CH₃, CF₃, M, and an amino or substituted amino group; R₂ can be selected from H, CH₃, and M; R₃, R₄, and R₅ can be each independently selected from H, OH, alkoxy, M, and an amino or substituted amino group; at least one boronic acid moiety is present; and M can be selected from Formula (II) and Formula (III), wherein L is an amino-containing linking moiety:

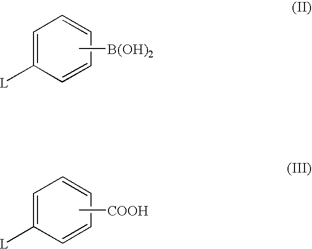

In some embodiments, the SMMR may include one or more compounds of Formulae (IV)-(X):

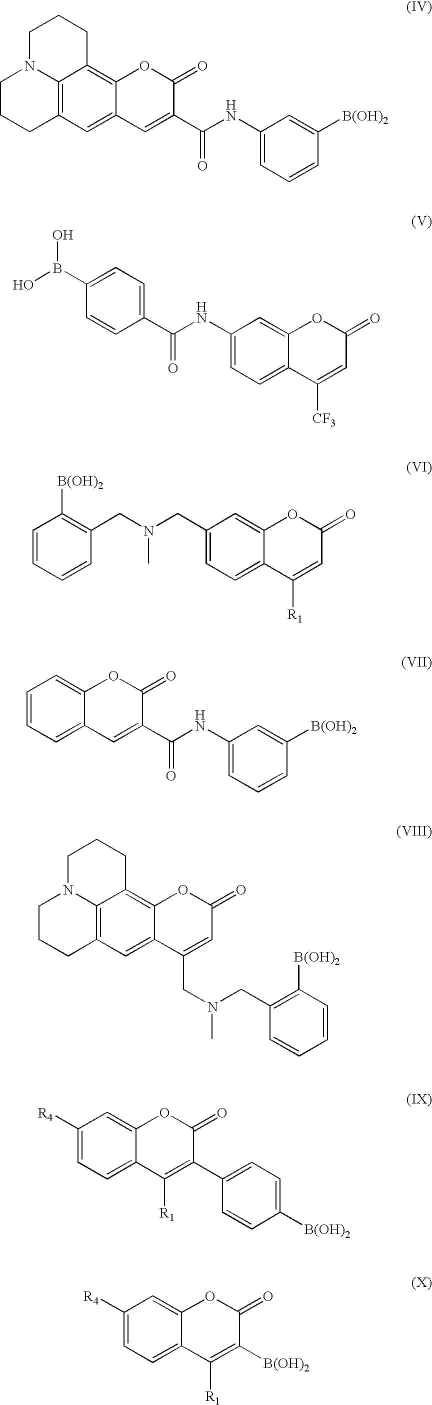

In some embodiments, a single species of SMMR is used. In other embodiments, multiple species of SMMRs are used. For example, a mixture of SMMRs of Formulae (V) and (VI) may be used. The SMMR may be administered as a pharmaceutical composition. Those of ordinary skill in the art will appreciate that the SMMR may be administered with one or more of a solvent, excipient, carrier, and diluent. In an embodiment, the SMMR may be modified with a molecular size attachment.

Additional SMMRs and methods for synthesis and design thereof that can be used in conjunction with the systems and methods herein are disclosed in U.S. patent application Ser. No. 11/349,731 and U.S. patent application Ser. No. 10/584,821, which is the United States national phase entry for PCT Appl. No. PCT/US2004/043087, incorporated by reference in their entireties herein. A particular SMMR can be derived on the basis of a combination of molecular properties including, but not limited to, specific molecular size, polarity, charge, structure, pKa, solubility, and the size and type of molecular attachments or anchors. Further guidelines for choosing or designing an appropriate SMMR can be found in PCT Appl. No. PCT/US2004/043087.

Another embodiment provides a pharmaceutical composition that can include one or more SMMRs described herein, and further including at least one selected from a pharmaceutically acceptable excipient, a carrier, and a diluent. In some embodiments, prodrugs, metabolites, stereoisomers, hydrates, solvates, polymorphs, and pharmaceutically acceptable salts of the compounds disclosed herein (e.g., the SMMR) are provided.

Disclosed herein are methods for delivering at least one SMMR into the human epidermis, which may include contacting the SMMR with an array of microneedles and penetrating the epidermis with the microneedles to a depth of about 10 μm, wherein the depth corresponds with the bottom of the dead stratum corneum layer, to about 175 μm, wherein the depth corresponds with the top of the dermal layer.

In some embodiments, an array of microneedles may be used. In some embodiments, the array of microneedles may be attached to a syringe or other apparatus suitable for the administration of at least one SMMR to a subject. In an exemplary embodiment, the base of the array can be attached to a handle or other means to assist contacting the microneedle array with the skin. In another embodiment, an array of hollow microneedles can be attached at the base to a coupling piece, wherein the coupling piece will couple the microneedles to a syringe.

A variety of methods may be used to contact the SMMR with the microneedle. In an embodiment, the tips of a microneedle array may be lined and/or coated with the SMMR. In some embodiments, the tips may be lined with the SMMR immediately before administration to the subject; in other embodiments, the tips may be pre-lined ahead of time and stored for later use.

In other embodiments, the microneedle tip may comprise at least one SMMR. In an exemplary embodiment, the SMMR tip may be added during the microneedle manufacture process by coating the silicon wafer or epoxy mold with an SMMR solution prior to etching. In another embodiment, an SMMR solution may be applied to the tip of a microneedle and allowed to evaporate, leaving behind solid SMMR.

In still other embodiments, the at least one SMMR can be applied directly to skin and the microneedle can be subsequently contacted with the treated area.

Those skilled in the art will appreciate that most areas of the skin are suitable for carrying out the methods disclosed herein. In some embodiments, the SMMR and microneedle are applied to an area of the skin wherein the stratum corneum layer is of average or less than average thickness. For example, the SMMR may be applied to a fingertip or arm. Less preferable areas include hands and feet where the stratum corneum layer is significantly thicker. In some embodiments, because the microneedle does not reach the dermis, it also does not reach the nerves, thus rendering administration nearly painless.

In some embodiments, force is applied to the microneedle to assist in penetration of the epidermis. In an exemplary embodiment, a force of approximately 10 N can be applied to a 20×20 microneedle array. In some embodiments, upon penetration of the stratum corneum, the SMMR dissolves in the water-based epidermis.

In some embodiments, a hollow microneedle can be used. In an embodiment, an array of hollow microneedles may be used, wherein each microneedle is filled with SMMR. As explained above, the base of such an array may be coupled to a syringe in some embodiments. After the microneedles penetrate the stratum corneum, the SMMR can be injected into the epidermis via the syringe.

Penetration of the SMMR through the stratum corneum may be accomplished further in conjunction with a variety of methods, including but not limited to passive and active diffusion. In some embodiments, passive diffusion may be used. In other embodiments, active diffusion may be used. Active diffusion methods include, for example, electroporation, laser poration, sonic poration, ultrasonic poration, iontophoresis, mechanical poration, solvent transport, and direct application by painting.

Disclosed herein are in vivo methods for monitoring the concentration of one or more metabolites or analytes, which may include applying at least one SMMR to at least one surface of a living tissue, organ, and/or whole organism for a predetermined period of time; causing penetration of the SMMR to a depth of about 10 μm to about 175 μm, into the epidermis, wherein the penetration is caused by a microneedle array; and monitoring a change in the concentration of the one or more metabolites or analytes in a metabolic pathway by detecting changes in the at least one SMMR at one or more time points using an optical reader.

The at least one SMMR is applied to the desired depth or location within skin. As described herein, the fluorescence mechanism used is either a direct or indirect indication of the glucose concentration in the target cell environment. In some embodiments, the quantification of the change in one or more of fluorescence emission, absorption, reflectance, and scattering of the SMMR can be monitored using fluorescence or absorption spectroscopy. Those of ordinary skill in the art will appreciate that any fluorescence or absorption spectroscopic techniques can be used in accordance with the methods disclosed herein. In an embodiment, the optical reader may calculate the skin response to glucose, apply first principles mathematical models to the response, and provide a determination of the blood glucose levels. Choosing the particular commercially available or custom designed optical reader that is compatible for use with the methods and compositions of this invention is within the ability of one skilled in the art. Additional methods for determining in vivo concentration of a metabolite and/or analyte are disclosed in U.S. patent application Ser. No. 11/349,731.

It will be understood by those of skill in the art that numerous and various modifications can be made without departing from the spirit of the present invention. Therefore, it should be clearly understood that the forms of the present invention are illustrative only and not intended to limit the scope of the present invention.

What is claimed is:

1. A system for delivering at least one small molecule metabolic reporter (SMMR) into the human epidermis, comprising:
at least one SMMR; and
a microneedle array;
wherein said microneedle array penetrates said epidermis to facilitate delivery of said SMMR.

2. The system of claim 1, wherein the at least one SMMR comprises a compound of Formula (I): 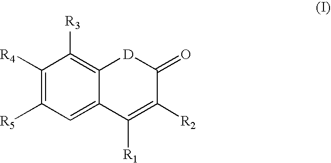
wherein D is a heteroatom;
R₁ is selected from the group consisting of H, OH, CH₃, CF₃, M, an amino group, and a substituted amino group;
R₂ is selected from the group consisting of H, CH₃, and M;
R₃, R₄, and R₅ are each independently selected from the group consisting of H, OH, alkoxy, M, an amino group, and a substituted amino group;
M is selected from the group consisting of Formula (II) and Formula (III): 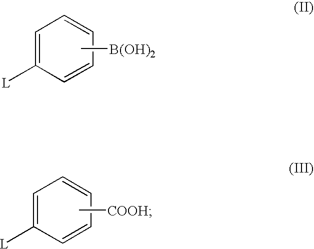
L, when present, is an amino-containing linking moiety; and
at least one boronic acid moiety is present.

3. An in vivo method for delivering at least one SMMR into the human epidermis, comprising:
contacting said SMMR with a microneedle array, and;
penetrating said epidermis with said array to a depth of about 10 μm, wherein said depth corresponds with the bottom of the dead stratum corneum layer, to about 175 μm, wherein said depth corresponds with the top of the dermal layer.

4. The method of claim 3, wherein the at least one SMMR comprises a compound of Formula (I): 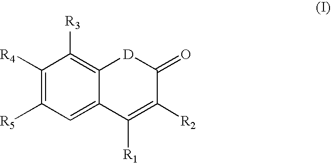
wherein D is a heteroatom;
R₁ is selected from the group consisting of H, OH, CH₃, CF₃, M, an amino group, and a substituted amino group;
R₂ is selected from the group consisting of H, CH₃, and M;
R₃, R₄, and R₅ are each independently selected from the group consisting of H, OH, alkoxy, M, an amino group, and a substituted amino group;
M is selected from the group consisting of Formula (II) and Formula (III): 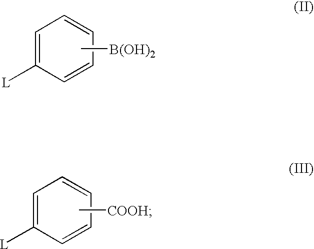
L, when present, is an amino-containing linking moiety; and
at least one boronic acid moiety is present.

5. The method of claim 3, wherein said microneedle array comprises solid microneedles.

6. The method of claim 5, wherein said contact between at least one SMMR and microneedle array comprises lining the array of microneedles with at least one SMMR.

7. The method of claim 5, wherein said contact between at least one SMMR and microneedle array comprises an array of microneedles with SMMR tips.

8. The method of claim 3, wherein said microneedle array comprises hollow microneedles.

9. The method of claim 8, wherein said contact between at least one SMMR and microneedle array comprises filling said hollow microneedles with at least one SMMR.

10. The method of claim 9, further comprising the step of injecting said at least one SMMR into the epidermis with a syringe.

11. The method of claim 3, wherein said contact between at least one SMMR and microneedle array comprises contacting said array with at least one SMMR applied directly to skin.

12. An in vivo method for monitoring the concentration of one or more metabolite or analyte, the method comprising:
contacting at least one SMMR with a microneedle array;
penetrating the human epidermis with said microneedles to a depth of about 10 μm, wherein said depth corresponds with the bottom of the dead stratum corneum layer, to about 175 μm, wherein said depth corresponds with the top of the dermal layer; and
monitoring a change in the concentration of the one or more metabolite or analyte in a metabolic pathway by detecting changes in the at least one SMMR at one or more time points using an optical reader.

13. The method of claim 12, wherein said one or more metabolite or analyte comprises glucose.

14. The method of claim 13, wherein the at least one SMMR comprises a compound of Formula (I): 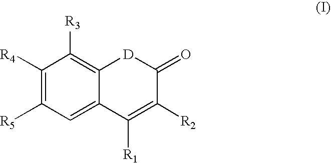
wherein D is a heteroatom;
R₁ is selected from the group consisting of H, OH, CH₃, CF₃, M, an amino group, and a substituted amino group;
R₂ is selected from the group consisting of H, CH₃, and M;
R₃, R₄, and R₅ are each independently selected from the group consisting of H, OH, alkoxy, M, an amino group, and a substituted amino group;
M is selected from the group consisting of Formula (II) and Formula (III): 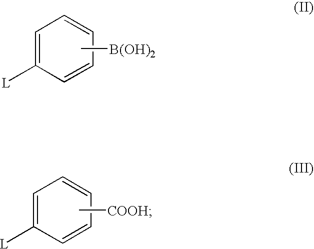
L, when present, is an amino-containing linking moiety; and
at least one boronic acid moiety is present.
